(19) United States
(12) Patent Application Publication
(10) Pub. No.: US 2004/0171947 A1
(43) Pub. Date: Sep. 2, 2004

(54) Pulse wave detecting apparatus

(75) Inventors: Toshihiko Ogura, Komaki-shi (JP); Kiyoyuki Narimatsu, Komaki-shi (JP)

Correspondence Address: OLIFF & BERRIDGE, PLC, P.O. BOX 19928, ALEXANDRIA, VA, 22320, US

(73) Assignee: COLIN MEDICAL TECHNOLOGY CORPORATION, Komaki-shi (JP)

(21) Appl. No.: 10/780,569

(22) Filed: Feb. 19, 2004

(30) Foreign Application Priority Data
Feb. 28, 2003 (JP) 2003-053640

(51) Int. Cl. A61B 5/02

(52) U.S. Cl. 600/500; 600/502

(57) ABSTRACT

A pulse wave detecting apparatus, including a pressure pulse wave sensor 30 which detects a pulse wave produced from a living subject and outputs a pulse wave signal SM representing the detected pulse wave, a signal converting device 78 which modulates, by using the pulse wave signal SM as a modulating signal, an audible frequency of a to-be-modulated signal, and thereby provides a modulated signal having audible frequencies, and a speaker 62 which outputs a sound representing the modulated signal provided by the signal converting device 78.

### BACKGROUND OF THE INVENTION

1. Field of the Invention

The present invention relates to a pulse wave detecting apparatus which detects a pulse wave produced from a living subject.

2. Related Art Statement

The shape of pulse wave produced from a living person is influenced by various diseases, such as arteriosclerosis or cardiomyopathy, and it has been proposed to use the shape of pulse wave to diagnose those diseases. This proposal is disclosed in, e.g., “Basic And Clinic Arterial Pulse Wave”, Yoshiaki Masuda & Hiroshi Kanai, First Edition, p. 28-p. 51, March, 2000, Kyoritsu Shuppan K.K., Japan. According to this document, the shape of pulse wave shows various changes resulting from arteriosclerosis and, first of all, a tidal wave is greater than a percussion wave.

When a detected pulse wave is used in making a diagnosis, it is conventional to output the detected pulse wave on a display device, or on a recording medium by a printer, so that a medical person such as a doctor can recognize the shape of the pulse wave outputted. By the way, in many cases, heart sounds and/or blood flow sounds are detected to make a diagnosis on a living person. In those cases, those sounds are outputted from a stethoscope or a speaker. The heart sounds are used to diagnose the condition of the heart; and the blood flow sounds are used to diagnose the condition of the blood vessel, such as arteriostenosis. Since the pulse wave reflects the condition of the heart and/or the blood vessel, it is desirable to output, like the heart sounds and/or the blood flow sounds, the pulse wave in the form of sound.

### SUMMARY OF THE INVENTION

It is therefore an object of the present invention to provide a pulse wave detecting apparatus which can output, as an audible sound, a pulse wave detected from a living subject.

Since a portion of a frequency band of a pulse wave detected from a living subject is lower than a frequency band audible by a human being. Therefore, to achieve the above object, it is needed to convert frequencies of a pulse wave detected by a pulse wave sensor into audible frequencies.

The above object has been achieved by the present invention. According to the present invention, there is provided a pulse wave detecting apparatus, comprising a pulse wave sensor which detects a pulse wave produced from a living subject and outputs a pulse wave signal representing the detected pulse wave; a signal converting means for converting, according to a predetermined relationship between magnitude of pulse wave signal, and frequency, the pulse wave signal outputted from the pulse wave sensor, into a converted signal having an audible frequency; and a sound outputting device which outputs a sound representing the converted signal provided by the signal converting means.

According to this invention, the signal converting means converts, according to the predetermined relationship between magnitude of pulse wave signal and frequency, the pulse wave signal outputted from the pulse wave sensor, into the converted signal having the audible frequency, and the sound outputting device outputs, as the sound, the converted signal provided by the signal converting means. Therefore, the frequency of the sound outputted from the sound outputting device changes with the magnitude of the pulse wave signal, i.e., a carotid pulse wave. Thus, a medical person such as a doctor can diagnose a disease, such as arteriosclerosis, based on the change of pitch of the sound outputted from the sound outputting device.

Here, preferably, the pulse wave detecting apparatus further comprises a signal normalizing means for normalizing a magnitude of the pulse wave signal outputted from the pulse wave sensor, and the signal converting means converts, according to the predetermined relationship, the pulse wave signal that has been normalized by the signal normalizing means, into the converted signal. According to this feature, the signal normalizing means normalizes the pulse wave signal outputted from the pulse wave sensor, into a normalized signal having a prescribed range of change. Therefore, even in the case where the absolute magnitude of the pulse wave signal outputted from the pulse wave sensor is small, the frequency of the sound outputted from the sound outputting device sufficiently largely changes. Thus, even if the magnitude of the pulse wave signal may be considerably small over its entire length, the medical person can accurately recognize the shape of the pulse wave, based on the change of pitch of the sound outputted from the sound outputting device.

Also, preferably, the signal converting means modulates, by using the pulse wave signal as a modulating signal, an audible frequency of a to-be-modulated signal, and thereby provides a modulated signal as the converted signal.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a diagrammatic view showing a circuitry of a carotid pulse wave detecting apparatus to which the present invention is applied;

FIG. 2 is an illustrative view showing a state in which a pressure pulse wave detecting probe of the apparatus of FIG. 1 is worn on a cervical portion of a living subject;

FIG. 3 is an enlarged view of the pressure pulse wave detecting probe of FIG. 2, a portion of the probe being cut away;

FIG. 4 is a view for explaining a state in which an array of pressure sensing elements is provided in a pressing surface of a pressure pulse wave sensor shown in FIG. 3;

FIG. 5 is a diagrammatic view for explaining essential control functions of an electronic control device of the apparatus of FIG. 1;

FIG. 6 is a flow chart for explaining the control functions of the control device, shown in FIG. 5; and

FIG. 7 is a graph showing an expression representing a linear relationship between magnitude of pressure pulse wave signal SM, and frequency.

### DETAILED DESCRIPTION OF PREFERRED EMBODIMENTS

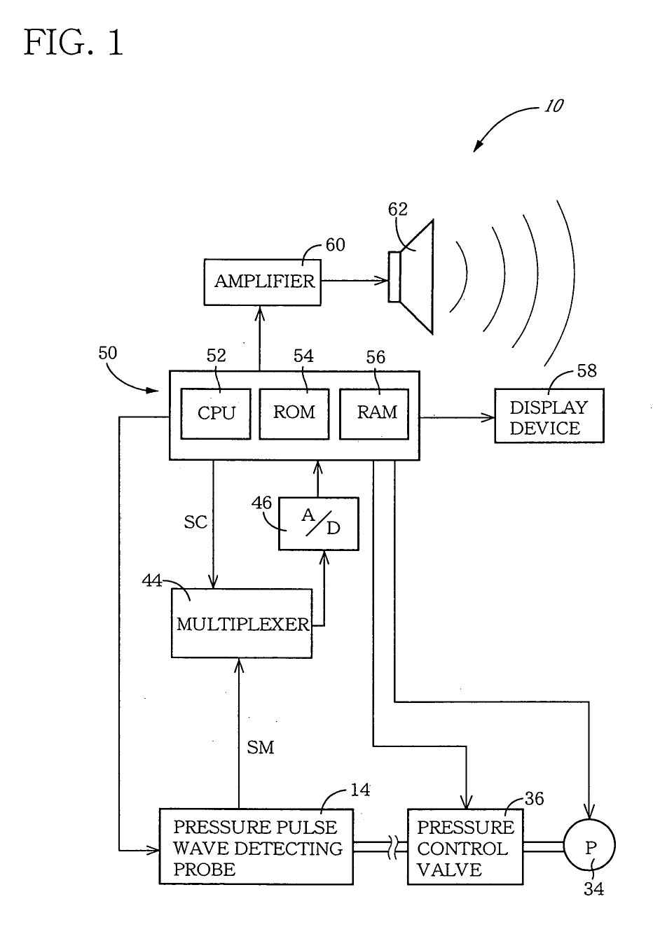

Hereinafter, there will be described a preferred embodiment of the present invention in detail by reference to the drawings. FIG. 1 is a diagrammatic view showing a circuitry of a carotid pulse wave detecting apparatus 10 to which the present invention is applied.

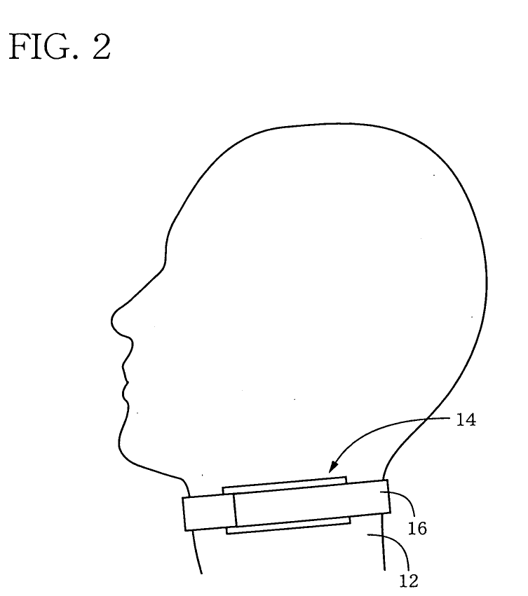

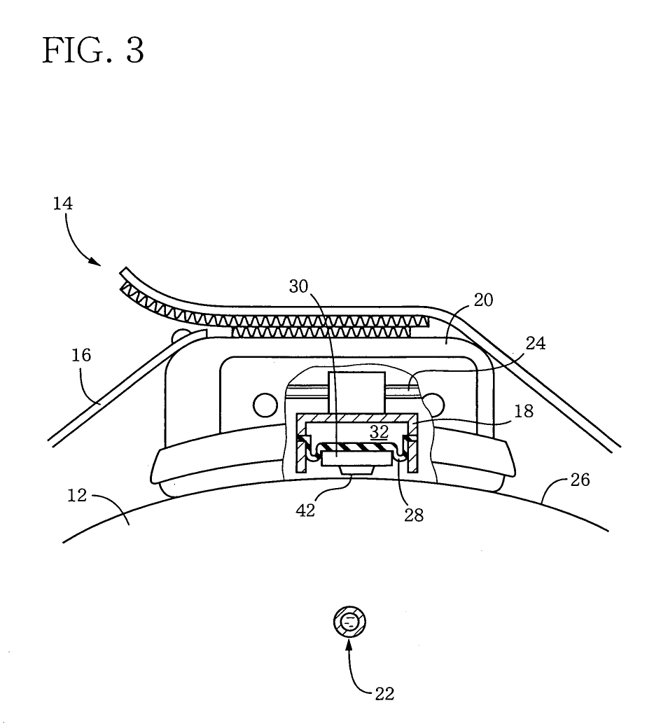

The carotid pulse wave detecting apparatus 10 includes a pressure pulse wave detecting probe 14. The pressure pulse wave detecting probe 14 is worn on a cervical portion 12 of a living subject, as shown in FIG. 2, with the help of a band 16, so as to detect non-invasively a carotid pulse wave as a pressure pulse wave that is produced from a carotid artery 22 (FIG. 3) of the subject. As shown in detail in FIG. 3, the pressure pulse wave detecting probe 14 includes a container-like sensor housing 18; a case 20 which accommodates the sensor housing 18; and a feed screw 24 which is threadedly engaged with the sensor housing 18 and is rotated by an electric motor, not shown, provided in the case 20 so as to move the sensor housing 18 in a widthwise direction of the carotid artery 22. With the help of the band 16, the pressure pulse wave detecting probe 14 is detachably attached to the cervical portion 12, such that an open end of the sensor housing 18 is opposed to a body surface 26 of the cervical portion 12.

In addition, the detecting probe 14 includes a pressure pulse wave sensor 30 which is secured via a diaphragm 28 to an inner wall of the sensor housing 18, such that the sensor 30 is movable relative to the housing 18 and is advanceable out of the open end of the same 18. The sensor housing 18, the diaphragm 28, etc. cooperate with each other to define a pressure chamber 32, which is supplied with a pressurized air from an air pump 34 via a pressure control valve 36, as shown in FIG. 1, so that the pressure pulse wave sensor 30 is pressed against the body surface 26 with a pressing force corresponding to the air pressure in the pressure chamber 32.

The sensor housing 18 and the diaphragm 28 cooperate with each other to provide a pressing device 38 which presses the pressure pulse wave sensor 30 against the carotid artery 22, and the feed screw 24 and the not-shown electric motor cooperate with each other to provide a pressing-position changing device or a widthwise-direction moving device 40 which moves the pressure pulse wave sensor 30 in the widthwise direction of the carotid artery 22 and thereby changes a pressing position where the sensor 30 is pressed on the body surface 26.

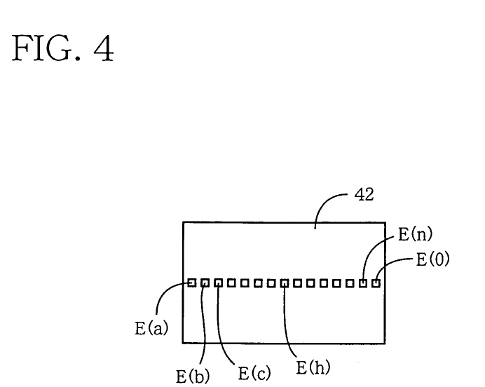

The pressure pulse wave sensor 30 has a pressing surface 42 defined by a semiconductor chip consisting of, e.g., a mono-crystalline silicon, and a number of semiconductor pressure-sensing elements (hereinafter, simply referred to as the “pressure sensing elements”) E which are arranged in the pressing surface 42 at a regular interval in the widthwise direction of the carotid artery 22, i.e., in the direction of movement of the sensor 30 parallel to the feed screw 24, over a length greater than the diameter of the carotid artery 22. For example, as shown in FIG. 4, fifteen pressure sensing elements E(a), E(b), . . . , E(o) are arranged at a regular interval of, e.g., 0.6 mm.

The pressure pulse wave detecting probe 14, constructed as described above, is pressed against the body surface 26 of the cervical portion 12 right above the carotid artery 22, so that each of the pressure sensing elements E of the pressure pulse wave sensor 30 detects a pressure pulse wave, i.e., a carotid pulse wave which is produced from the carotid artery 22 and is transmitted to the body surface 26, and supplies a pressure pulse wave signal SM representing the detected carotid pulse wave, to a multiplexer 44, as shown in FIG. 1.

The multiplexer 44 receives the respective pressure pulse wave signals SM outputted from the fifteen pressure sensing elements E of the pressure pulse wave sensor 30, and supplies, according to switch signals SC supplied thereto from an electronic control device 50, described later, the fifteen pressure pulse wave signals SM, sequentially in a prescribed order and each signal for a prescribed time duration, to an A/D (analog to digital) converter 46, which converts each of the pressure pulse wave signals SM into a digital signal and supplies the digital signal to the control device 50.

The electronic control device 50 is provided by a so-called microcomputer including a CPU (central processing unit) 52, a ROM (read only memory) 54, a RAM (random access memory) 56, and an I/O (input and output) port, not shown. The CPU 52 processes signals according to the control programs pre-stored in the ROM 54 by utilizing the temporary-storage function of the RAM 56, and supplies drive signals via respective drive circuits, not shown, to the air pump 34 and the pressure control valve 36 so as to control the air pressure in the pressure chamber 32. In addition, the CPU 52 operates the widthwise direction moving device 40 to change the pressing position where the pressure pulse wave sensor 30 is pressed on the body surface 26 of the cervical portion 12. Moreover, the CPU 52 supplies, to the multiplexer 44, the switch signals SC at a prescribed period that is sufficiently shorter than an average pulse period, and normalizes the pressure pulse wave signal SM continuously supplied from the multiplexer 44 via the A/D converter 46. In addition, the CPU 52 operates a display device 58 to display the thus normalized pressure pulse wave signal SM, converts the normalized signal SM into a signal having audible frequencies, and the thus converted signal to an amplifier 60, which amplifies the converted signal and supplies the amplified signal to a speaker 62 functioning as a pulse sound outputting device. The speaker 62 outputs a pulse sound of a carotid pulse wave.

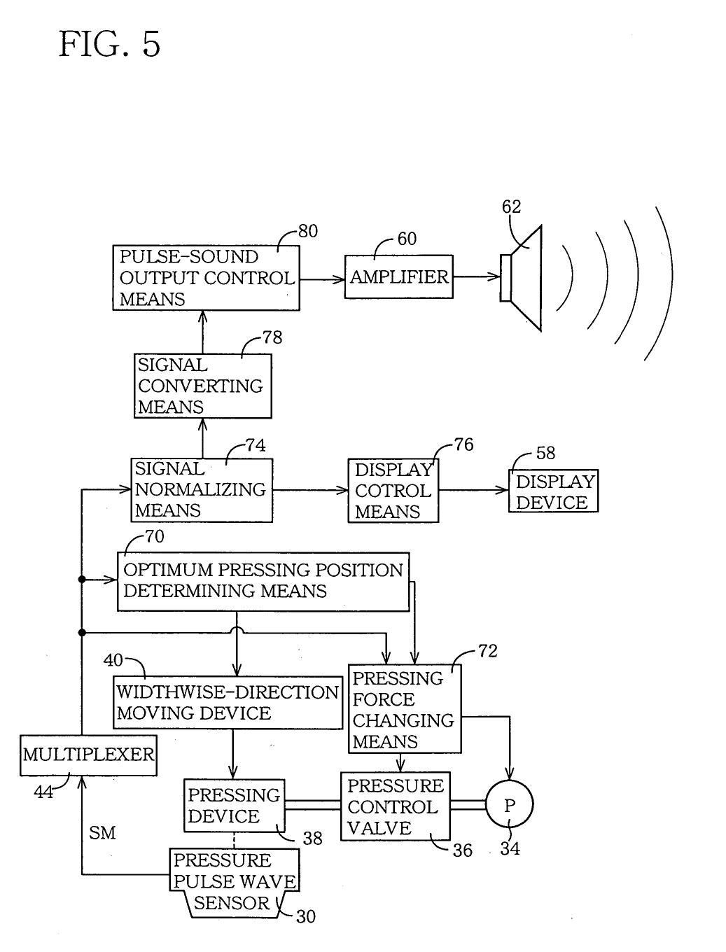

FIG. 5 is a diagrammatic view for explaining essential control functions of the electronic control device 50 of the pulse wave detecting apparatus 10. In FIG. 5, an optimum pressing position determining device or means 70 determines an optimum pressing position where the pressure pulse wave sensor 30 is pressed on the body surface 26 of the cervical portion 12. More specifically described, the determining means 70 determines judges whether a prescribed pressing position changing condition is satisfied, i.e., whether one of the fifteen pressure sensing elements E of the sensor 30 that detects the highest one of the respective pressure values detected by all the elements E (hereinafter, referred to as the “highest pressure detecting element EM) is positioned in one of prescribed opposite end portions of the array of elements E. Each of the prescribed opposite end portions of the array of elements E may be a range having a prescribed length including a corresponding one of the opposite ends of the array of elements E, or a range accommodating a prescribed number of elements E including a corresponding one of the respective elements E located at the opposite ends of the array. When this pressing position changing condition is satisfied, e.g., when the sensor 30 is initially worn on the subject, the optimum pressing position determining means 70 carries out the following pressing position changing operation: After the pressing device 38 once moves the pressure pulse wave sensor 30 away from the body surface 26, the widthwise direction moving device 40 moves the pressing device 38 and the sensor 30 over a prescribed distance, and then the pressing device 38 again presses the sensor 30 with a prescribed, considerably small first pressing force, HDP1, that would be sufficiently lower than a diastolic blood pressure of the artery (i.e., the carotid artery 2) to which the sensor 30 is to be pressed. In this state, the determining means 70 judges again whether the prescribed pressing position changing condition is satisfied. The determining means 70 repeats carrying out the above described operation and judgment till the pressing position changing condition is not satisfied any longer, preferably till the highest pressure detecting element EM is positioned in a prescribed middle portion of the array of elements E. The length, or element number, employed for each of the opposite end portions of the array of elements E is prescribed based on the diameter of the artery 22 to be pressed by the sensor 30, and is, e.g., one fourth of the diameter.

A pressing force changing device or means 72 changes, after the optimum pressing position determining means 70 positions the pressure pulse wave sensor 30 at the optimum pressing position, a pressing force HDP (i.e., a hold-down pressure) applied by the pressing device 38 to the sensor 30, within a prescribed pressing force range, either stepwise in response to each heartbeat of the subject or continuously at a prescribed, considerably low rate. Based on the carotid pulse wave detected while the pressing force HDP is changed, the changing means 72 determines an optimum pressing force HDPO, and maintains the pressing force HDP of the pressing device 38, at the thus determined optimum pressing force HDPO. For example, a pressing force HDP of the pressing device 38 at the time when a pulse pressure of the carotid pulse wave detected by the highest pressure detecting element EM is greater than a prescribed threshold value is determined as the optimum pressing force HDPO. The pulse pressure is a difference between the highest pressure and the lowest pressure of one-heartbeat length of the carotid pulse wave. If the threshold value is too low, an unclear carotid pulse wave may be detected by the highest pressure sensing element EM. Hence, the threshold value is experimentally prescribed at such a value which assures that a clear carotid pulse wave can be detected.

A signal normalizing device or means 74 normalizes a magnitude of the pressure pulse wave signal SM that is continuously detected by the highest pressure detecting element EM in the state in which the pressing force changing means 72 maintains the pressing force HDP applied to the pressure pulse wave sensor 30, at the optimum pressing force HDPO, in such a way that an amplitude of each of successive heartbeat synchronous pulses of the carotid pulse wave represented by the pressure pulse wave signal SM is made equal to a prescribed reference value. The amplitude of each heartbeat synchronous pulse is a difference between the greatest magnitude and the smallest magnitude of the each pulse. Thus, the signal normalizing means 74 provides a normalized pulse wave signal SMn.

A display control device or means 76 controls the display device 58 to display the normalized pulse wave signal SMn provided by the signal normalizing means 74, i.e., a normalized carotid pulse wave. The shape or waveform of the carotid pulse wave outputted by the display device. 58 can be used by a medical person such as a doctor to diagnose a disease of the subject, e.g., arteriosclerosis. In the present embodiment, however, the shape of the carotid pulse wave is outputted in the form of a sound as well, as described below.

A signal converting device or means 78 continuously converts, according to a predetermined relationship between magnitude of normalized pulse wave signal SMn, and frequency, the normalized pulse wave signal SMn provided by the signal normalizing means 74, into a signal having audible frequencies, i.e., an audible sound signal f(t). To this end, the technique of frequency modulation known in the communication technology is utilized in the present embodiment. More specifically, the above-indicated relationship is represented by the following expression 1 to modulate a to-be-modulated signal using the normalized signal SMn as a modulating signal:

f(t)=A cos {ω_{c}t+Φ_{b}+ω_{d}∫₀^{t}SMn(t)dt} (Expression 1)

where t is time; A is amplitude; ω_{c} is angular frequency of the to-be-modulated signal; Φ₀ is integration constant; and ω_{d} is proportion constant, and those are experimentally determined.

The angular frequency ω_{c} of the to-be-modulated signal is an audible frequency (e.g., 10 KHz); and the constant ω_{d} is so determined that the range of change of the audible sound signal f(t) is sufficiently large.

In the case where Expression 1 representing the predetermined relationship is used, the greater the magnitude of normalized pulse wave signal SMn is, the higher the frequency of audible sound signal f(t) is and, the smaller the magnitude of normalized pulse wave signal SMn is, the lower the frequency of audible sound signal f(t) is.

A pulse-sound output control device or means 80 supplies the audible sound signal f(t) provided by the signal converting means 78, that is, a signal that has audible frequencies and represents a pulse sound of the carotid pulse wave, to the amplifier 60, so that the speaker 62 outputs the pulse sound of the carotid pulse wave that is audible to the medical person.

The pulse sound of the carotid pulse wave, outputted from the speaker 62, has the feature that the greater the normalized pulse wave signal SMn(t) is, the higher frequency the pulse sound has and, the smaller the normalized signal SMn(t) is, the lower frequency the pulse sound has. Therefore, the medical person can recognize the shape of the carotid pulse wave, based on the change of pitch of the sound outputted from the speaker. More specifically described, since a carotid pulse wave having a normal shape and a carotid pulse wave having an abnormal shape provide respective sounds having different changes of pitch, the medical person can judge whether the shape of carotid pulse wave is normal or abnormal, based on the change of pitch of the sound outputted by the speaker 62. For example, the medical person can find, based on the sound outputted from the speaker 62, a disease that changes the shape of pulse wave, for example, arteriosclerosis.

A carotid pulse wave detected from a normal person has a clear dicrotic notch. Therefore, the magnitude of a carotid pulse wave continues decreasing between a maximum point thereof and a dicrotic notch thereof, subsequently increases and then slowly decreases. Thus, the sound outputted from the speaker 62 first shows a high pitch representing the maximum point, and subsequently the pitch lowers, then increases, and gradually lowers. In contrast, a carotid pulse wave detected from a patient suffering arteriosclerosis has an unclear dicrotic notch; and a carotid pulse wave detected from a patient suffering advanced arteriosclerosis does not have the dicrotic notch. In the last case, the pitch of the sound outputted from the speaker 62 monotonously lowers after the high pitch representing the maximum point, and does not increase any more.

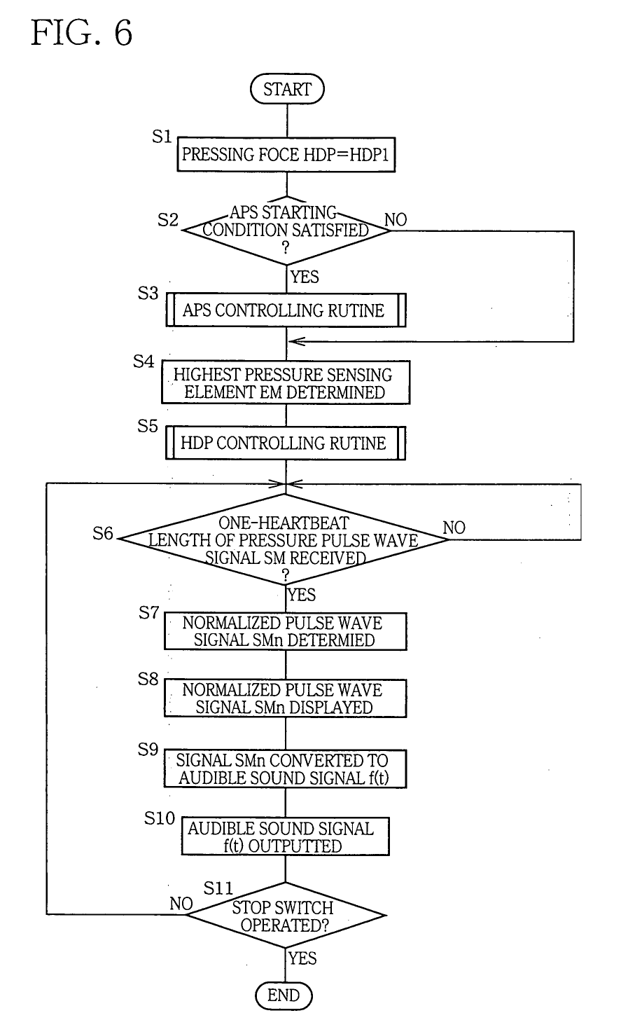

FIG. 6 is a flow chart representing the control functions of the electronic control device 50, shown in the diagrammatic view of FIG. 5. While implementing this flow chart, the control device 50 periodically supplies the switch signals SC to the multiplexer 44, and stores, in the RAM 56, the pressure pulse wave signals SM sequentially supplied from the multiplexer 44.

First, the control device carries out Steps S1, S2, and S3 corresponding to the optimum pressing position determining means 70. At Step S1, the control device operates the air pump 34 and the pressure control valve 36 to change the pressure in the pressure chamber 32 so that the pressing force HDP applied to the pressure pulse wave sensor 30 is changed to the prescribed first pressing force HDP1 that would be sufficiently lower than the diastolic blood pressure of the carotid artery 22.

Subsequently, the control of the control device goes to Step S2 where the control device judges whether the prescribed pressing position changing condition (i.e, an APS starting condition) is satisfied, i.e., whether the highest pressure sensing element EM of the pressure pulse wave sensor 30 is positioned in one of the prescribed opposite end portions of the array of pressure sensing elements E. If a negative judgment is made at Step S2, the control jumps to Step S4 and the following steps, described later.

On the other hand, if a positive judgment is made at Step S2, that is, if the position of the pressure pulse wave sensor 54 relative to the carotid artery 22 is not appropriate, the control goes to Step S3 to carry out an APS controlling routine. More specifically described, the highest pressure sensing element EM is moved to an optimum pressing position where the highest pressure sensing element EM is located at substantially the middle of the array of pressure sensing elements E, in such a manner that after the pressing device 38 once moves the pressure pulse wave sensor 30 away from the body surface 26, the widthwise direction moving device 40 moves the pressing device 38 and the sensor 30 over a prescribed distance, and then the pressing device 38 again presses the sensor 30 with the above described first pressing force HDP1. In this state, the control device judges again whether the highest pressure sensing element EM is positioned in a prescribed middle portion of the array of elements E. The control device repeats carrying out those operation and judgment till a positive judgment is made at Step S3.

After the pressure pulse wave sensor 30 is positioned at the optimum pressing position at Step S3, or if a negative Judgment is made at Step S2, then the control goes to Step S4. At Step S4, the control device determines a highest pressure sensing element EM that detects the highest pressure in this state. Subsequently, the control goes to Step S5 corresponding to the pressing force changing means 72. At Step S5, the control device implements an HDP controlling routine. More specifically described, the control device operates the pressing device 38 to press the pressure pulse wave sensor 30 while continuously increasing the pressing force HDP from the first pressing force HDP1, and determines, as an optimum pressing force HDPO, a value of the pressing force HDP at the time when the pulse pressure of carotid pulse wave detected by the highest pressure sensing element EM, determined at Step S4, during the increasing of the pressing force HDP, first exceeds the prescribed threshold value. Then, the control device maintains the pressing force HDP applied to the pressure pulse wave sensor 30, at the thus determined optimum pressing force HDPO.

Subsequently, at Step S6, the control device judges whether the control device has received a one-heartbeat length of the pressure pulse wave signal SM, after the last positive judgment was made at this step S6. In a special case where Step S6 is carried out for the first time after the commencement of this routine, the control device judges whether it has received a one-heartbeat length of the pressure pulse wave signal SM, after the commencement of this routine. The control device repeats Step S6 till a positive judgment is made at this step. Step S6 is followed by Step S7 corresponding to the signal normalizing means 74. At Step S7, the control device normalizes the one-heartbeat length of the pressure pulse wave signal SM provided by the highest pressure sensing element EM determined at Step S4, such that the amplitude of the one-heartbeat length of the signal SM is made equal to the prescribed reference value, and thereby provides a normalized pulse wave signal SMn.

Subsequently, the control goes to Step S8 corresponding to the display control means 76. At Step S8, the control device operates the display device 58 to display the normalized pulse wave signal SMn obtained at Step S7, i.e., a normalized carotid pulse wave.

Then, the control goes to Step S9 corresponding to the signal converting means 78. At Step S9, the control device converts, using the above-indicated Expression 1, the one-heartbeat length of the normalized pulse wave signal SMn, obtained at Step S7, into the audible sound signal f(t) having the angular frequency ω_{c} as the center frequency. Subsequently, the control goes to Step S10 corresponding to the pulse-sound output control means 80. At Step S10, the control device supplies the audible sound signal f(t) obtained at Step S9, to the amplifier 60, so that the speaker 62 outputs the sound that has the audible frequencies and represents the carotid pulse wave.

Subsequently, at Step S11, the control device judges whether a stop switch, not shown, has been operated to stop the operation of the present apparatus 10. If a negative judgment is made at Step S11, the control goes back to Step S6 and the following steps so as to continue displaying an image representing the carotid pulse wave, on the display 58, and outputting a sound representing the carotid pulse wave, from the speaker 62. Meanwhile, if a positive judgment is made at Step S11, the control device quits this routine.

In the above-described embodiment, the signal converting means 78 (Step S9) converts, using Expression 1, the pressure pulse wave signal SM provided by the highest pressure sensing element EM of the pressure pulse wave sensor 30, into the audible sound signal f(t), such that the greater the magnitude of the pulse wave signal SM is, the higher audible frequency the audible sound signal f(t) has, and the speaker 62 outputs the thus obtained audible sound signal f(t). Therefore, the frequency of the sound outputted from the speaker 62 changes with the magnitude of the pulse wave signal, i.e., a carotid pulse wave. Thus, a medical person such as a doctor can diagnose a disease, such as arteriosclerosis, based on the change of pitch of the sound outputted from the speaker 62.

In addition, in the above-described embodiment, the signal normalizing means 74 (Step S7) normalizes the pressure pulse wave signal SM outputted from the pressure pulse wave sensor 30, into the normalized signal having the prescribed range of change. Therefore, even in the case where an absolute magnitude of the pulse wave signal SM outputted from the pulse wave sensor 30 is small, the frequency of the sound outputted from the speaker 62 sufficiently largely changes. Thus, even if the magnitude of the pulse wave signal SM may be considerably small over its entire length, the medical can accurately recognize the shape of the carotid pulse wave, based on the change of pitch of the sound outputted from the speaker 62.

While the present invention has been described in its preferred embodiment by reference to the drawings, it is to be understood that the invention may otherwise be embodied.

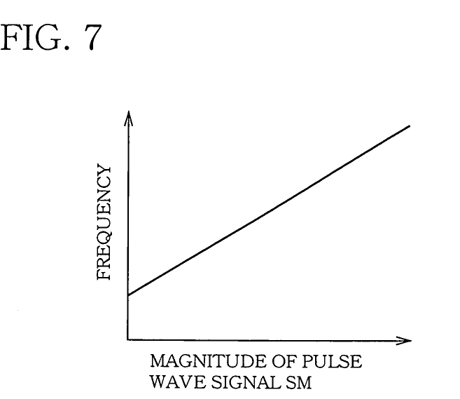

For example, in the above-described embodiment, the signal converting means 78 converts the pressure pulse wave signal SM into the audible sound signal f(t), by using the relationship represented by the above-indicated Expression 1 to modulate the audible frequency of the to-be-modulated signal using the pulse wave signal SM. However, the signal converting means 78 may be modified to convert the pressure pulse wave signal SM into an audible sound signal, by using a predetermined linear relationship between magnitude of pressure pulse wave signal SM and frequency, shown in FIG. 7.

In addition, in the above-described embodiment, the signal normalizing means 74 normalizes the pressure pulse wave signal SM provided by the pressure pulse wave sensor 30, and the signal converting means 78 converts the normalized pulse wave signal SMn into the audible sound signal f(t). However, it is possible not to employ the signal normalizing means 74. In the latter case, the signal converting means 78 may be modified to directly convert the pressure pulse wave signal SM into the audible sound signal f(t). In this case, if the absolute magnitude of the pressure pulse wave signal SM is small, the range of change of frequency of the sound outputted from the speaker 62 is also small, and accordingly it is more difficult for the medical person to recognize the shape of the carotid pulse wave than in the above-described embodiment. On the other hand, based on the range of change of frequency, and an average pitch, of the sound outputted from the speaker 62, the medical person can recognize an average magnitude of the pressure pulse wave signal SM provided by the sensor 30. Therefore, the medical person can know how the sensor 30 is worn on the body surface 26 of the subject, for example, the position where the sensor 30 is worn, and the pressing pressure with which the sensor 30 is pressed on the body surface 26.

In addition, the above-described pulse wave detecting apparatus 10 employs, as the pulse wave sensor, the pressure pulse wave sensor 30 of the type which has, in the pressing surface 42, the array of pressure sensing elements E each of which detects, as the pressure pulse wave, the carotid pulse wave. However, it is possible to employ a different type of pulse wave sensor, e.g., a semiconductor capacitor type pressure sensor in which a silicon diaphragm is interposed between upper and lower electrodes; or a volumetric pulse wave sensor which detects a volumetric pulse wave. The volumetric pulse wave sensor may be a photoelectric pulse wave detecting probe for use in blood oxygen saturation measurement, or a photoelectric pulse wave sensor which is adapted to be worn on a tip of a finger to detect, e.g., pulsation of a living subject.

In addition, in the above-described embodiment, the pulse wave is detected from the cervical portion 12 of the subject. However, the pulse wave may be detected from a different portion of the subject, such as a brachium.

In addition, in the above-described embodiment, the speaker 62 functions as the sound outputting device. However, the speaker 62 may be replaced with an earphone or a headphone. Moreover, the audible sound signal may be supplied to the sound outputting device via a wire or wireless communication.

It is to be understood that the present invention may be embodied with other changes and improvements that may occur to a person skilled in the art without departing from the spirit and scope of the invention defined in the appended claims.

What is claimed is:

1. A pulse wave detecting apparatus, comprising:
a pulse wave sensor which detects a pulse wave produced from a living subject and outputs a pulse wave signal representing the detected pulse wave;
a signal converting means for converting, according to a predetermined relationship between magnitude of pulse wave signal, and frequency, the pulse wave signal outputted from the pulse wave sensor, into a converted signal having an audible frequency; and
a sound outputting device which outputs a sound representing the converted signal provided by the signal converting means.

2. The apparatus according to claim 1, further comprising a signal normalizing means for normalizing a magnitude of the pulse wave signal outputted from the pulse wave sensor,
wherein the signal converting means converts, according to the predetermined relationship, the pulse wave signal that has been normalized by the signal normalizing means, into the converted signal.

3. The apparatus according to claim 1, wherein the signal converting means modulates, by using the pulse wave signal as a modulating signal, an audible frequency of a to-be-modulated signal, and thereby provides a modulated signal as the converted signal.

4. The apparatus according to claim 1, further comprising a display device which displays the pulse wave signal outputted from the pulse wave sensor.

5. The apparatus according to claim 2, further comprising a display device which displays the pulse wave signal that has been normalized by the signal normalizing means.

6. A pulse wave detecting apparatus, comprising:
a pulse wave sensor which detects a pulse wave produced from a living subject and outputs a pulse wave signal representing the detected pulse wave;
a signal converting device which converts, according to a predetermined relationship between magnitude of pulse wave signal, and frequency, the pulse wave signal outputted from the pulse wave sensor, into a converted signal having an audible frequency; and
a sound outputting device which outputs a sound representing the converted signal provided by the signal converting device.

7. The apparatus according to claim 6, further comprising a signal normalizing device which normalizes a magnitude of the pulse wave signal outputted from the pulse wave sensor,
wherein the signal converting device converts, according to the predetermined relationship, the pulse wave signal that has been normalized by the signal normalizing device, into the converted signal.
